(19) United States
(12) Patent Application Publication
(10) Pub. No.: US 2019/0151630 A1
(43) Pub. Date: May 23, 2019

(54) BALLOON FOR MEDICAL USE AND METHOD FOR MANUFACTURE OF BALLOON CATHETER

(71) Applicant: OLYMPUS CORPORATION, Tokyo (JP)

(72) Inventor: Yuri TAKEUCHI, Tokyo (JP)

(73) Assignee: OLYMPUS CORPORATION, Tokyo (JP)

(21) Appl. No.: 16/257,962

(22) Filed: Jan. 25, 2019

Related U.S. Application Data
(63) Continuation of application No. PCT/JP2017/029094, filed on Aug. 10, 2017.

(30) Foreign Application Priority Data
Sep. 6, 2016 (JP) 2016-174039

(51) Int. Cl. A61M 25/10; A61L 29/14; A61M 25/00

(52) U.S. Cl. CPC A61M 25/1029; A61L 29/14; A61L 29/085; A61M 25/1034; A61M 25/005

(57) ABSTRACT

A balloon for medical use includes a polyamide elastomer and a polyamide resin.

The application is a continuation application based on a PCT Patent Application No. PCT/JP2017/029094, filed Aug. 10, 2017, whose priority is claimed on Japanese Patent Application No. 2016-174039, filed Sep. 6, 2016. The content of both the PCT Application and the Japanese Applications are incorporated herein by reference.

### FIELD OF THE INVENTION

The present invention relates to technology suitably used for a balloon for medical use and a method for manufacture of a balloon catheter.

### DESCRIPTION OF RELATED ART

Some balloon catheters (balloon dilators) for medical use are used to dilate a narrowed area of a lumen in a living body such as an esophagus, a bile duct, a blood vessel, or the like.

Such a balloon catheter is configured such that a diameter thereof increases in response to an applied pressure within a range of working pressure on design.

In the balloon catheter, two characteristics that (1) a response of an expansion amount (compliance) to application (increase) of a pressure to be able to reliably release narrowing is fast, and (2) pressure resistant strength is high so that expansion is safe for a living body are desirable.

In the balloon catheter, a polyamide elastomer or the like has been used since the past as disclosed in Japanese Unexamined Patent Application, First Publication No. 2013-146505.

### SUMMARY OF THE INVENTION

A balloon for medical use of a first aspect of the present invention includes a polyamide elastomer and a polyamide resin.

According to a balloon for medical use of a second aspect of the present invention, in the first aspect, at least one of the polyamide elastomer and the polyamide resin may be crosslinked.

A method for manufacturing a balloon for medical use of a third aspect of the present invention is a method for manufacturing the balloon for medical use according to the first aspect, and includes a parison forming process of kneading the polyamide elastomer and the polyamide resin, and forming a parison from a kneaded product of the polyamide elastomer and the polyamide resin, and a blow molding process of blow molding the parison in a die.

The method for manufacturing the balloon for medical use of a fourth aspect of the present invention, in the third aspect, further may include a crosslinking process of crosslinking at least one of the polyamide elastomer and the polyamide resin.

A method for manufacturing the balloon for medical use of a fifth aspect of the present invention, in the fourth aspect, may have the crosslinking process in which a crosslinker selected from a group consisting of carbodiimide-based, acid anhydride-based, isocyanate-based, and oxazoline-based materials is used.

A balloon catheter of a sixth aspect of the present invention includes the balloon for medical use according to the first aspect.

### BRIEF DESCRIPTION OF THE DRAWINGS

FIG. 1 is a schematic view showing a first embodiment of a balloon catheter according to the present invention.

FIG. 2 is an axial schematic sectional view showing a first embodiment of a balloon for medical use according to the present invention.

FIG. 3 is an axial sectional view showing a die in a first embodiment of a method for manufacturing a balloon for medical use according to the present invention.

### DETAILED DESCRIPTION OF THE INVENTION

Hereinafter, a first embodiment of a balloon for medical use, a balloon catheter, and a method for manufacturing a balloon for medical use according to the present invention will be described with reference to the drawings.

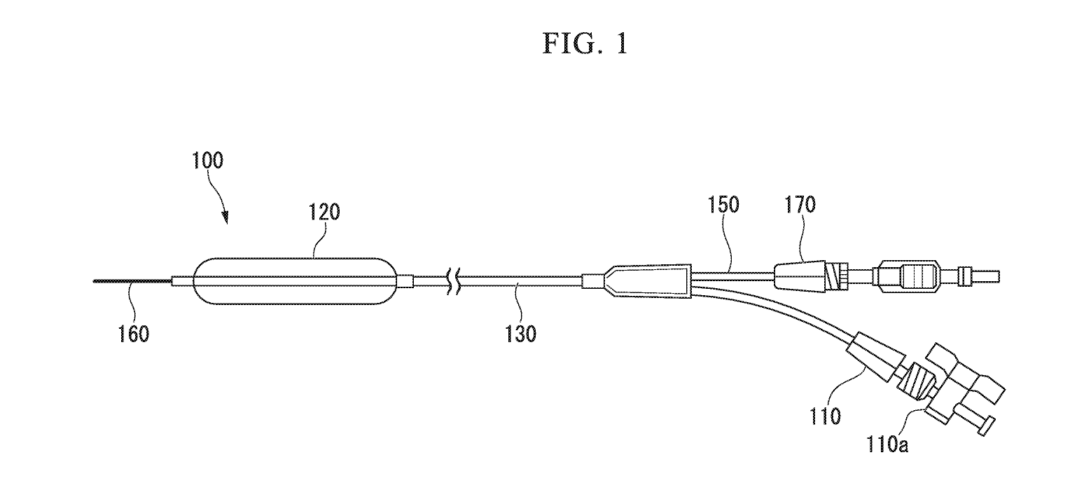

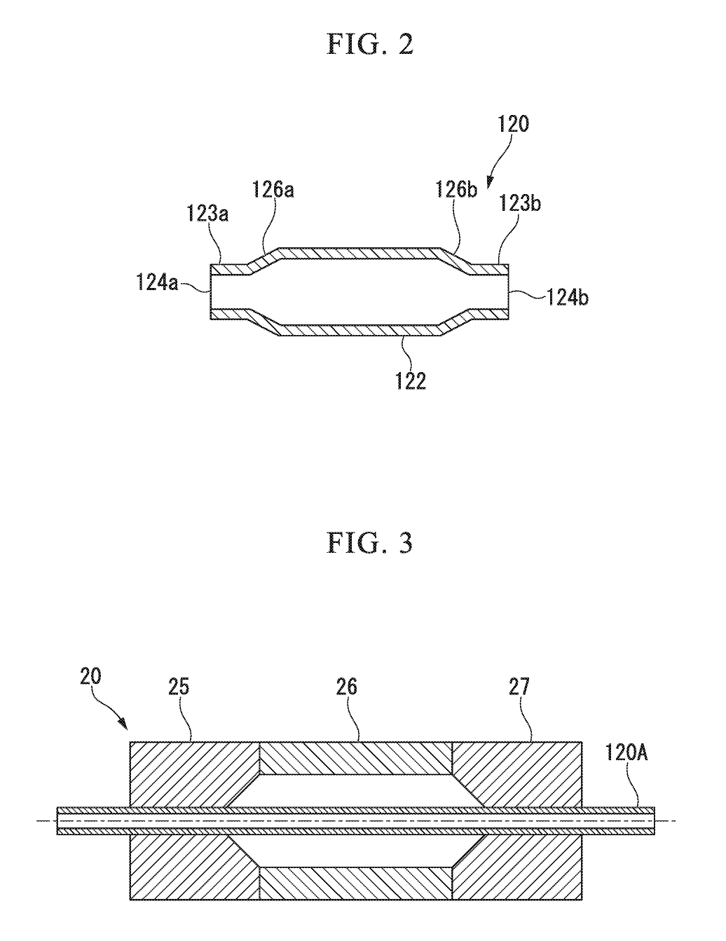

FIG. 1 is a schematic view showing a balloon catheter having a balloon for medical use in the present embodiment, and FIG. 2 is a sectional view showing a balloon for medical use in the present embodiment. In FIG. 1, a reference sign 100 indicates a balloon catheter.

As shown in FIG. 1, a balloon catheter 100 in the present embodiment has a hub 110, a balloon (a balloon for medical use) 120, a proximal shaft 130, and a guide wire lumen tube 150 as a main body.

The hub 110 is disposed near a hand of a medical doctor who manipulates the balloon catheter 100 (on a distal side). A stop cock 110a is provided on the hub 110, and the hub 110 is configured to be connectable to a pressure application device (not shown) such as an inflator that supplies a high-pressure fluid.

The proximal shaft 130 is joined to the hub 110 to extend to a distal side in a fluid-communicable way, and the balloon 120 is joined to a distal side of the proximal shaft 130.

A flow passage for supplying the high-pressure fluid into the balloon 120 is formed inside the proximal shaft 130.

A proximal end of the balloon 120 serves as a connector 123b (to be described below), surrounds an outer circumferential surface of the proximal shaft 130, and is joined to the outer circumferential surface. A distal end of the balloon 120 serves as a connector 123a (to be described below), surrounds the outer circumferential surface, and is joined to the outer circumferential surface of the proximal shaft 130. Alternatively, the distal end of the balloon 120 may serve as the connector 123a (to be described below), surround an outer circumferential surface of the guide wire lumen tube 150 that serves as a distal shaft, protrudes from a distal side of the proximal shaft 130, and be joined to the outer circumferential surface of the guide wire lumen tube 150.

The high-pressure fluid supplied to the balloon 120 stays in the balloon 120 due to an opening (not shown) of the proximal shaft 130 provided inside the balloon 120, and the balloon 120 is dilated. That is, before the high-pressure fluid is supplied into the balloon 120, the balloon 120 is folded nearly in the same dimension as an outer diameter of the proximal shaft 130, and is provided to come into close contact with the outer circumferential surface of the proximal shaft 130 on the distal side of the proximal shaft 130. When the high-pressure fluid is supplied into the balloon 120, the folds unfold, and thereby the balloon 120 is dilated. In FIG. 1, the state in which the balloon 120 is dilated is shown.

The guide wire lumen tube 150 is provided to pass through the proximal shaft 130 to form a coaxial type or biaxial type double tube structure without a lumen (a guide wire lumen) thereof communicating with a fluid supply flow passage of the proximal shaft 130 and to further pass through the balloon 120. An opening of the guide wire lumen tube 150 on the distal side of the guide wire lumen tube 150 is arranged further on the distal side than a distal portion of the balloon 120. An opening of the guide wire lumen tube 150 on the proximal side of the guide wire lumen tube 150 is provided as a guide wire port 170 that is an inserting/leaving port of the guide wire 160.

As shown in FIG. 2, the balloon 120 of the present embodiment is made up of a tubular membranous body 122 that can be dilated/contracted by the fluid supplied from the proximal shaft 130, and the connectors 123a and 123b that extend from opposite ends of the membranous body in an axial direction and are connected to the catheter.

The membranous body 122 is formed in a tubular shape having a nearly uniform outer diameter for dilating a narrowing area of a lumen in a living body such as a blood vessel, a urinary duct, a bile duct, or the like. Tapered parts 126a and 126b are formed such that shapes of opposite ends of the tubular membranous body 122 become tapered shapes.

Opposite ends of the tapered parts 126a and 126b which are formed in the tapered shapes are connected to the connectors 123a and 123b. Openings 124a and 124b are formed in the connectors 123a and 123b of the opposite ends, and the proximal shaft 130 is inserted therethrough.

The opening 124b of one of the connectors is formed in a larger diameter than the opening 124a of the other connector. The shapes of the tapered parts and the connectors may be different.

The balloon 120 of the present embodiment is made into a sac-like member formed of a single layer resin that contains a polyamide elastomer and a polyamide resin. In the resin of which the balloon 120 is formed, at least one of the polyamide elastomer and the polyamide resin may be crosslinked. A crosslinker is selected from carbodiimide-based, anhydride-based, isocyanate-based, and oxazoline-based materials. However, mixtures of other materials are not precluded. In this way, the material is crosslinked to increase molecular weight, and thereby resisting pressure performance can be further improved while maintaining compliance performance. The carbodiimide-based material is particularly desirable in view of biocompatibility, reactivity, and temporal stability, but an excessive crosslinking reaction remarkably damages extrusion moldability. In addition, since the excessive crosslinking reaction hinders an oriented crystal during blow molding, sufficient resisting pressure cannot be obtained. An addition amount is desirably less than or equal to a functional group equivalent.

The balloon 120 of the present embodiment is configured such that a diameter thereof increases in response to an applied pressure within a range of working pressure on design, and simultaneously satisfies two characteristics that (1) a response of an expansion amount (compliance) to application (increase) of a pressure is fast, and (2) pressure resistant strength is high. Particularly among balloons used to dilate a narrowing area of a lumen in a living body such as an esophagus, a bile duct, a blood vessel or the like, in the case where the balloon is used for the esophagus, it is required that compliance is high to be able to reliably release narrowing, and a conventional problem that the resisting pressure is low can be solved.

In this way, a balloon having high compliance and high resisting pressure is realized by using a mixture of the polyamide elastomer and the polyamide resin as a material of the balloon 120 in the present embodiment. The polyamide elastomer makes a great contribution to a high compliance characteristic, and the polyamide resin makes a great contribution to high resisting pressure.

As a result of an experiment, it was found that, for example, a polyether block amide is preferably used as the polyamide elastomer, and polyamide 11 or polyamide 12 is preferably used as the polyamide resin in view of achieving both the high compliance and the high resisting pressure, and they are excellent in compatibility.

Further, orientation is controlled so that a degree of orientation is increased with respect to a radial direction of the balloon 120 of the present embodiment. Thereby, the compliance performance of the balloon 120 can be made equal to or greater than that of the case where a balloon is formed of only a polyamide elastomer used as a base material, and the resisting pressure performance can simultaneously be made higher than that of the base material.

In general, when a polymeric material is oriented, strength increases in an orientation direction to be resistant to expansion. Therefore, since a cylindrical molding oriented in a radial direction is improved in strength, that is, pressure resistance, but is resistant to expansion, the degree of orientation in the radial direction is adjusted, so that the pressure resistance and expansibility can be adjusted.

Here, orientation of the resin can be realized by arranging a flow direction of the resin through stretching, pushing-in, leading-in, or the like. For example, when the resin in a molten state or a flexible state is stretched by pulling in one direction under applied pressure, directions of molecules of the resin are arranged in the direction of the stretching, and a degree of orientation in the direction of the stretching is improved. In the present embodiment, as will be described below, in a blow molding process of molding the balloon with respect to the parison made of the above material, an oriented state of the balloon is controlled. It was found by an experiment that, when a molecular weight is made higher by crosslinking, the degree of orientation is further improved even at the same stretching rate.

<Method for Manufacturing a Balloon for Medical Use>

Hereinafter a method for manufacturing a balloon for medical use of the present embodiment will be described.

The method for manufacturing a balloon for medical use of the present embodiment has a parison forming process and a blow molding process.

In the parison forming process, a polyamide elastomer and a polyamide resin are kneaded and extruded to form a single layer parison.

From the point of view of achieving both high compliance and a high resisting pressure, polyether block amide having shore hardness of 65D or higher is desirable as the polyamide elastomer, and polyamide 11 or polyamide 12 is desirable as the polyamide resin. Polyamide 1010, polyamide 1012, or metaxylenediamine (MXD) may be used as the polyamide resin.

In this case, the method may include a crosslinking process of crosslinking at least one of the polyamide elastomer and the polyamide resin.

In the crosslinking process, a crosslinker selected from a group consisting of carbodiimide-based, acid anhydride-based, isocyanate-based, and oxazoline-based chain extenders is used. In this case, the crosslinker crosslinks by heat during kneading.

Crosslinking is performed by reacting with these chain extenders, and is performed by solid-phase polymerization under a vacuum high-temperature environment. Furthermore, an epoxy crosslinker may be used as the crosslinker.

In the parison forming process, two kinds of materials are melted, kneaded, and compatibilized, and then are formed into a tube shape (a parison). At the time of the kneading, a crosslinking reaction is performed using the aforementioned crosslinker, and thereby molecular weight is made higher. An inner diameter and wall thickness of the parison are adjusted to become desired balloon compliance and wall thickness.

The parison forming process of forming the tubular parison using a stretchable polymer can be performed by a general-purpose extruding machine in which a die is mounted. A resin for molding is heated and melted to 180 to 300° C. in the extruding machine, is extruded from the die, and a single layer tubular parison is molded. An extruding temperature at this time is not particularly limited if it is a temperature at which the polymer can be melted, and is preferably 180 to 300° C., and more preferably 200 to 280° C.

In the blow molding process, the parison formed by the parison forming process is subjected to blow molding by a die.

FIG. 3 is a schematic sectional view showing a die used to manufacture the balloon for medical use in the present embodiment.

In the blow molding process, the parison is inserted into the die, is scaled at opposite ends thereof, and is heated in a state in which an internal pressure is applied.

To be specific, the parison 120A is inserted into the die 20 shown in FIG. 3, and blocks one end of the parison 120A. The blockage is performed by using heating and melting, seal caused by a high frequency, forceps, and so on. A heater that is heating means (not shown) and a cooling pipe that is cooling means are disposed outside the die 20. The die 20 is made up of separatable dies 25, 26 and 27. An inner surface shape formed when the separatable dies 25, 26 and 27 are combined is a basic outer surface shape of the balloon to be formed.

As shown in FIG. 3, the heater is operated to heat the parison 120A of a portion where the balloon 120 is formed to a temperature that is higher than or equal to a glass transition point of the polymer (the polyamide resin and the polyamide elastomer of which the tube is formed) and is lower than a melting point of the polymer, preferably a temperature ranging from 35 to 140° C., and more preferably a temperature ranging from 40 to 80° C. A gas is sent from the outside into the parison 120A while being pressurized, and the parison 120A is maintained for a fixed time in a heated and pressurized state. Then, the parison 120A is stretched in a direction in which the parison 120A extends (in a leftward/rightward direction of FIG. 3), is expanded at an internal pressure to bring the parison 120A of a portion heated in the die 20 into close contact with inner wall surfaces of the separatable dies 25, 26 and 27, and is molded in a balloon shape.

A pressure when the gas is introduced into the parison 120A is preferably 1.0 to 3.5 MPa, and more preferably 1.5 to 3.0 MPa.

Further, a distance when the parison 120A is stretched in the leftward/rightward direction of FIG. 3 is preferably 10 to 100 mm, more preferably 40 to 90 mm, and most preferably 50 to 80 mm.

At least one of the polyamide elastomer and the polyamide resin is crosslinked to increase molecular weight, and thereby the resisting pressure can be increased while maintaining the compliance.

The internal pressure is held for a fixed time while being applied at a temperature that is higher than or equal to a molding temperature and is lower than or equal to a melting point of the polymer, and a shape is memorized by performing annealing. Then, a coolant circulates in the cooling pipe, and the parison 120A is cooled at room temperature. Here, the parison may be cooled without annealing, or the parison may be cooled after being contracted at a temperature that is lower than or equal to the molding temperature. The cooling may be performed by simply leaving the parison as it is and natural cooling without circulation of the coolant. Afterward, the inside of the parison 120A is set to normal pressure, and the parison 120A is removed from the inside of the die 20. The parison 120A is cut at distal and proximal portions of the parison 120A, and thereby a basic shape of the balloon as shown in FIGS. 1 and 2 is formed.

The balloon catheter 100 of the present embodiment is inserted into a narrowing region, and performs dilation treatment of the balloon 120. In this case, a fluid supplied from the catheter to the balloon includes known fluids such as a contrast agent, helium gas, a physiological saline solution, CO₂ gas, O₂ gas, N₂ gas, air, and so on.

The balloon 120 is a foldable balloon, and can be kept folded on an outer circumference of the proximal shaft 130 in the state in which it is not dilated. The balloon 120 is a foldable balloon that has a tubular main body of nearly the same diameter in which at least a part becomes a cylindrical shape to make it possible to easily dilate the narrowing area such as an esophagus, a blood vessel or the like. The connector 123b of the balloon 120 is liquid-tightly fixed to the distal portion of the proximal shaft 130 by an adhesive or thermal fusion bonding. Likewise, the connector 123a is also liquid-tightly fixed to the distal portion of the proximal shaft 130.

The balloon 120 forms a space between an inner surface of the balloon 120 and an outer surface of the proximal shaft 130 during dilation. This space communicates with the outside through the opening of the proximal shaft 130. In this way, since the lumen of the proximal shaft 130 is made to communicate with the balloon 120, the fluid is easily injected into the balloon 120 by the lumen of the proximal shaft 130.

In the present embodiment, the balloon catheter 100 having the guide wire has been described, but can also be configured to have another member as the distal shaft without the guide wire 160.

### EXAMPLES

Hereinafter, examples according to the present invention will be described.

The balloon was formed of at least two kinds of materials, a first material of which was a polyamide elastomer (TPA) formed of polyether block amide, and a second material of which was a polyamide resin such as polyamide (PA) 11 or 12. At least one of the first material and the second material was crosslinked by reacting with a carbodiimide-based, an anhydride-based, an isocyanate-based, or an oxazoline-based chain extender, but was crosslinked to increase molecular weight by solid-phase polymerization under a vacuum high-temperature environment.

A die for a balloon having an inner diameter of ϕ12 mm was prepared, and a parison was expanded in the die for the balloon. Thereby, the balloon was molded. In this case, an inner diameter of the parison was changed, and thereby a stretching rate (a diameter increasing rate) in a radial direction during molding was adjusted to control orientation.

The resin and the diameter increasing rate were adjusted to manufacture the balloon, and a resisting pressure and a compliance rate were measured. The results are represented in Table 1.

TABLE 1

| | | Comparative | | | |
|---|---|---|---|---|---|
| | | Example | Example 1 | Example 2 | Example 3 |
| Resin | TPA | 100 | 100 | 100 | 100 |
| | PA12 | — | 50 | 100 | 100 |
| Crosslinker | Carbodiimide compound | — | — | — | 3.0 |
| Molecular weight ×10⁴ | | — | — | 2.8 | 4.8 |
| Balloon | Pressure when ruptured | 10.5 | 11.3 | 11.4 | 12.5 |
| characteristics | (resisting pressure) atm | | | | |
| | Outer diameter when ruptured mm | 16.5 | 17.0 | 17.2 | 18.0 |
| | Compliance rate mm/atm | 0.6 | 0.6 | 0.6 | 0.6 |

An internal pressure was applied at a fixed rate to the balloon until the balloon was ruptured, and an outer diameter at each 1.5 atm after 3.0 atm and at the time of rupture was measured. In this case, in Comparative Example, an outer diameter at the time of pressurization of 3.0 atm was ϕ12 mm, and a balloon was pressurized and expanded, and was ruptured at 016.5 mm at the time of pressurization of 10.5 atm. However, in Example 1, an outer diameter at the time of pressurization of 3.0 atm was ϕ12 mm, and a balloon was ruptured at ϕ17.0 mm at the time of pressurization of 11.3 atm.

In Example 2, an outer diameter at the time of pressurization of 3.0 atm was ϕ12 mm, and a balloon was ruptured at +17.2 mm at the time of pressurization of 11.4 atm.

Example 3, an outer diameter at the time of pressurization of 3.0 atm was 412 mm, and a balloon was ruptured at ϕ18.0 mm at the time of pressurization of 12.5 atm.

In Comparative Example that is the same as the related art in which polyamide is not mixed, it is found from the results that the resisting pressure is low.

Thereby, by adding a polyamide resin a polyamide elastomer, and by making molecular weight higher, the resin is improved in strength and is easily stretched and oriented. By making an inner diameter of the parison is made greater than when the polyamide elastomer is used as a solo substance, while keeping compliance is equal to or greater than the balloon formed of the solo substance of the polyamide elastomer, the resisting pressure performance can be increased.

According to the present invention, the balloon and the balloon catheter having excellent manipulability and safety can be provided.

While preferred embodiments of the invention have been described and illustrated above, it should be understood that these are exemplary of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims.

What is claimed is:

1. A balloon for medical use, the balloon comprising:
a polyamide elastomer; and
a polyamide resin.

2. The balloon for medical use according to claim 1,
wherein at least one of the polyamide elastomer and the polyamide resin is crosslinked.

3. A method for manufacturing the balloon for medical use according to claim 1, the method comprising:
a parison forming process of kneading the polyamide elastomer and the polyamide resin, and forming a parison from a kneaded product of the polyamide elastomer and the polyamide resin; and
a blow molding process of blow molding the parison in a die.

4. The method for manufacturing the balloon for medical use according to claim 3, further comprising a crosslinking process of crosslinking at least one of the polyamide elastomer and the polyamide resin.

5. The method for manufacturing the balloon for medical use according to claim 4,
wherein a crosslinker selected from a group consisting of carbodiimide-based, acid anhydride-based, isocyanate-based, and oxazoline-based materials is used in the crosslinking process.

6. A balloon catheter comprising the balloon for medical use according to claim 1.
